(19) United States
(12) Patent Application Publication
(10) Pub. No.: US 2006/0271035 A1
(43) Pub. Date: Nov. 30, 2006

(54) Bipolar tissue dessication system and method

(75) Inventors: William K. Wheeler, Alamo, CA (US); Eric K.Y. Chan, San Carlos, CA (US)

Correspondence Address: FITCH EVEN TABIN AND FLANNERY, 120 SOUTH LA SALLE STREET, SUITE 1600, CHICAGO, IL, 60603-3406, US

(73) Assignee: Cardima, Inc.

(21) Appl. No.: 11/442,024

(22) Filed: May 26, 2006

Related U.S. Application Data
(60) Provisional application No. 60/685,551, filed on May 27, 2005.

(51) Int. Cl. A61B 18/14

(52) U.S. Cl. 606/41

(57) ABSTRACT

A first member of a clamping device is positioned at a first side of a portion of human tissue. The first member has a plurality of electrodes disposed thereupon. A second member of the clamping device is positioned at a second and opposite side of the human tissue. The second member has a plurality of thermocouples disposed thereupon. The first member is aligned with the second member such that each of the thermocouples on the second member is positioned across the tissue from and in the general vicinity of at least one electrode for recording the temperature of the tissue adjacent to the at least one electrode. RF energy is supplied to the plurality of electrodes and a return electrical path is provided from the plurality thermocouples. The path is electrically isolated from the RF energy supplied to the plurality of electrodes and terminates at an ablation monitoring and RF energy delivery system. The RF energy delivery system responsively adjusts an amount of RF energy delivered to the plurality of electrodes based upon the temperature.

### CROSS-REFERENCE TO RELATED APPLICATIONS

This application claims the benefit of Provisional Application Ser. No. 60/685,551 filed May 27, 2005, which is incorporated herein by reference in its entirety.

### FIELD OF THE INVENTION

The field of the invention relates to surgical methods and devices and, more specifically, to surgical methods and devices that utilize clamps or clamping mechansims.

### BACKGROUND OF THE INVENTION

Ablation is an example of a surgical procedure in which radio frequency (RF) energy is used to treat certain abnormalities of human tissue, for example, cardiac tissue. In one example, ablation procedures are used to treat cardiac fibrillation of cardiac tissue. During the RF ablation of cardiac tissue, deep lesions need to be created in the cardiac tissue while, at the same time, avoiding coagulum formation. In other words, RF energy must be delivered efficiently to the tissue, but not delivered and lost into the blood stream of the patient.

Generally, in ablation systems, radio frequency energy is delivered to the tissue by the use of RF energy generators and delivered by the generators to the tissue in two phases. Initially, a “ramp up” phase occurs during which time a relatively high amount of power is delivered to an ablating electrode until a desired set temperature (or some other property such as impedance) is sensed by a thermocouple or an electrode, respectively. Next, in a “regulation” phase of energy delivery, power is still delivered to the tissue, but this power is regulated at a lower level in order to maintain a desired target temperature. This target temperature is predetermined by the operator, and is generally set to 50° to 80° C. for the ablation of cardiac tissue. The temperature of the tissue, sensed by the thermocouples, may be fed back a controller, which is used to perform the regulation.

Various types of devices may be used to perform ablation procedures. For instance, single surgical catheters have been used. In another example, clamping devices that clamp or hold tissue between two jaws or members have also been utilized to perform ablation. In all of these devices, the electrodes and thermocouples are intermixed and placed in close proximity to each other so that the RF energy can be applied to the electrodes and feedback readings taken by the thermocouples.

Unfortunately, the accuracy and operation of current systems is limited because of electrical interference caused by the proximate placement of the electrodes and the thermocouples. Because of this interference, inaccurate feedback readings can occur and these readings may present false or misleading information to an operator or to a controller. The inaccurate information provided to the operator or controller may, in turn, cause inaccurate adjustments to be made to the amount and other characteristics of the applied RF energy, thereby resulting in inadequate treatment or potential damage of the tissue.

### SUMMARY OF THE INVENTION

A system and method for ablating human tissue using a clamp that separates RF energy emitting electrodes and thermocouples (or temperature sensing element) from each other on separate members. In so doing, electrical interference between the thermocouples and the electrodes is minimized or eliminated, producing more accurate feedback readings and substantially eliminating improper adjustments made to the RF energy delivered to tissue.

In many of these embodiments, a first member of a clamping device is positioned at a first side of a portion of human tissue. The first member has a plurality of RF energy emitting electrodes disposed thereupon. A second member of the clamping device is positioned at a second and opposite side of the human tissue. The second member has a plurality of thermocouples (or temperature sensing elements) disposed thereupon. The first member may be aligned with the second member such that each of the thermocouples on the second member is positioned across the tissue from and in the general vicinity of at least one electrode for recording the temperature of the tissue adjacent to the at least one electrode. RF energy is supplied to the plurality of electrodes and a return electrical path is provided. The path is electrically isolated from the RF energy supplied to the plurality of electrodes and terminates at an ablation monitoring and RF energy delivery system. The ablation monitoring and RF energy delivery system responsively adjusts an amount of RF energy delivered to the plurality of electrodes based upon the temperature.

The first and second members may be aligned in a variety of configurations. For example, they may be aligned in a C-clamp arrangement or a scissors configuration. The alignment may also be structured as a mechanical configuration where the first and second members are substantially parallel and opposite from each other.

Temperature information (or other information such as impedance information) may be obtained from the plurality of thermocouples (or any other temperature sensing elements such as thermistors) and provided to an evaluation system. Other information (e.g., tissue capacitance information) can be derived from the sensed information. This information can then be used to make adjustments to the RF energy provided to the electrodes.

The thermocouples may be positioned or configured in a number of different ways. For instance, the thermocouples may be positioned on the exterior of the second member so as to come in direct contact with the portion of the human tissue. Other examples of electrode placement are possible.

Thus, a device is provided that minimizes the interference caused by the close placement of electrodes and thermocouples in ablation and other surgical devices. Consequently, accurate readings may be made and provided to monitoring systems and accurate adjustments may be made to the RF energy applied to human tissue.

### BRIEF DESCRIPTION OF THE DRAWINGS

FIG. 1 is a diagram of a system for providing ablation to tissue according to the present invention;

FIG. 2 is side view of a clamping device showing thermocouples and electrodes according to the present invention;

FIG. 3a is diagram of a scissors-configuration clamping device according to the present invention; and

FIG. 3b is a diagram of a C-clamp-configuration clamping device according to the present invention.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. It will further be appreciated that certain actions and/or steps may be described or depicted in a particular order of occurrence while those skilled in the art will understand that such specificity with respect to sequence is not actually required. It will also be understood that the terms and expressions used herein have the ordinary meaning as is accorded to such terms and expressions with respect to their corresponding respective areas of inquiry and study except where specific meanings have otherwise been set forth herein.

### DETAILED DESCRIPTION OF THE PREFERRED EMBODIMENTS

The description that follows relates to clamping devices that are used in dessication systems such as ablation systems. However, it will be realized that the approaches discussed herein can be applied to other surgical procedures and are not limited to ablation techniques. For example, these devices can be used to dessicate tissue in atrial anatomical sites to create transmural lesions following a MAZE-like procedure for treating atrial fibrillation. In addition, many of the examples discussed herein relate to performing ablation procedures on cardiac tissue. However, it will be understood that the approaches discussed herein can be applied to any type of tissue and are not limited to cardiac tissue. In addition, the procedures described herein could be performed orthoscopically, or alternatively, by making an incision on a patient and positioning the device through the incision (e.g., open heart surgery). Other surgical techniques or combinations of techniques may also be used. It will also be realized that the target temperature, in one example, 50° to 80° C. for the ablation of cardiac tissue, may be set to any appropriate value. Furthermore, although the following description describes the temperature sensing element as a thermocouple, it will be realized that the thermocouples may be exchanged with any temperature sensing element or device such as thermistors or any similar device or combination of devices.

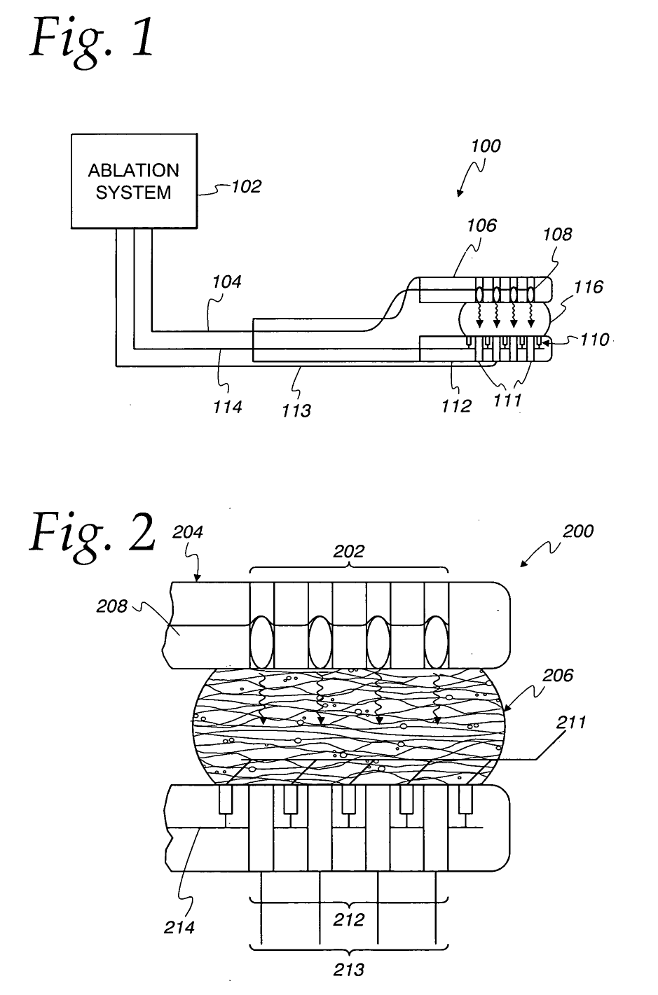

Referring now to FIG. 1, one example of a system for performing ablation on tissue is described. An ablation device 100 comprises two members 106 and 112. The two members are coupled to each other, for example, by use of a pivot or, in another example, as part of a sliding C-clamp arrangement, such that the members 106 and 112 are fitted about a portion of tissue 116.

The upper member 106 includes a plurality of RF energy emitting electrodes 108. The electrodes 108 may be coiled electrodes or any other type of electrodes. In addition, the electrodes and/or thermocouples can be formed by etching. The purpose of the electrodes is to provide RF energy for ablating the tissue 116. The RF energy is supplied to the electrodes by a wire or group of wires 104. The wire 104 is connected to an ablation system 102. As mentioned, the wire may be a single wire such that the electrodes are connected in series. In another approach, each electrode may be connected to a separate wire such that the wire 104 is a bundle of wires. In this later case, each of the wires in the bundle is insulated from and electrically isolated from each of the other wires.

The lower member 112 includes a plurality of thermocouples 110 and return electrodes 111. The thermocouples 110 are temperature sensors that provide temperature information (or other types of information such as impedance information) to the ablation system 102 via a return path 114, which may be a wire or bundle of wires. Alternatively, any other type of temperature sensing device such as thermistors may be used. The thermocouples 110 may be constructed from any type of suitable metal, and, in one example, are constructed of gold. The thermocouples may be placed or positioned on the surface of the member 112 so that they come into direct contact with the tissue 116. The thermocouples 110 may have a separate electrical paths 113 that return sensed temperature (e.g., voltage) information to the ablation system 102. The sensed information can be processed and other types of information (e.g., tissue capacitance information) can be derived from the sensed information.

The ablation system 102 is any suitable system that monitors and controls the RF energy applied to the tissue 116 via the electrodes 108. In one example, the ablation system is an INTELLITEMP® system manufactured by Cardima, Inc. Other examples of ablation systems may also be used.

As will be discussed elsewhere in this specification, the electrodes 108 and thermocouples 110 may be aligned so that they are substantially directly across from each other. The thermocouples 110 may be positioned between the return electrodes 111 or within the return electrodes 111. In some of these embodiments, the members 106 and 112 are formed or arranged so that these members are substantially parallel with each other. The return path 114 of the return electrodes 111 is electrically isolated from the RF energy path 104 that is used to supply RF energy to the electrodes on the first member. In other words, in a preferred approach, a bipolar clamping arrangement is formed. Consequently, the electrodes 108 and the thermocouples 110 are positioned such that electrical interference between the electrodes 108, return electrodes 111, and thermocouples 110 is reduced to a minimum or eliminated.

In one example of the operation of the system of FIG. 1, the first member 106 is aligned with the second member 112 such that each of the thermocouples 110 and/or return electrodes 111 on the second member 112 are positioned across the tissue 116 from and in the general vicinity of at least one of the electrodes 108 for recording the temperature of the tissue 116 adjacent to one of the electrodes 108. The device may be maneuvered through the body around portions of an organ, such as the heart. RF energy is supplied to the electrodes 108 and a return electrical path 114 is provided from the return electrodes 111. The path 114 is electrically isolated from the RF energy supplied to the electrodes 108 and terminates at the ablation monitoring and RF energy delivery system 102. The RF energy delivery system 102 responsively adjusts an amount of RF energy delivered to the electrodes 108 based upon the temperature.

Referring now to FIG. 2, one example of a clamping device 200 for providing RF energy to RF energy emitting electrodes 202 is described. An upper member 204 includes a plurality of RF energy emitting electrodes 202. The electrodes 202 are used to provide RF energy to a portion of tissue 206. The electrodes 202 are coupled together by a wire or bundle of wires 208 that supplies the RF energy to the electrodes 202.

The clamping device 200 also includes a lower member 210. The upper member 204 and lower member 210 may be aligned such that they are substantially parallel to each other. However, it will be understood that in alternative approaches (such as by using a scissors-like device) the members may not be substantially parallel to each other.

The lower member 210 has a plurality of thermocouples 212 that are aligned substantially directly across from one of the electrode 202. However, it will be understood that two or more electrodes may be aligned to one thermocouple and that two or more thermocouples may be aligned to a single electrode. In addition, only a majority of the electrode-thermocouple pairs may be aligned and a few of the pairs may be unaligned. In addition, the lower member 210 has a plurality of return electrodes 211.

The thermocouples 212 are provided with a return paths 213. The return electrodes 211 have a common return path 214 that is electrically isolated from the electrical path 208 that is used to provide RF energy to the electrodes 202.

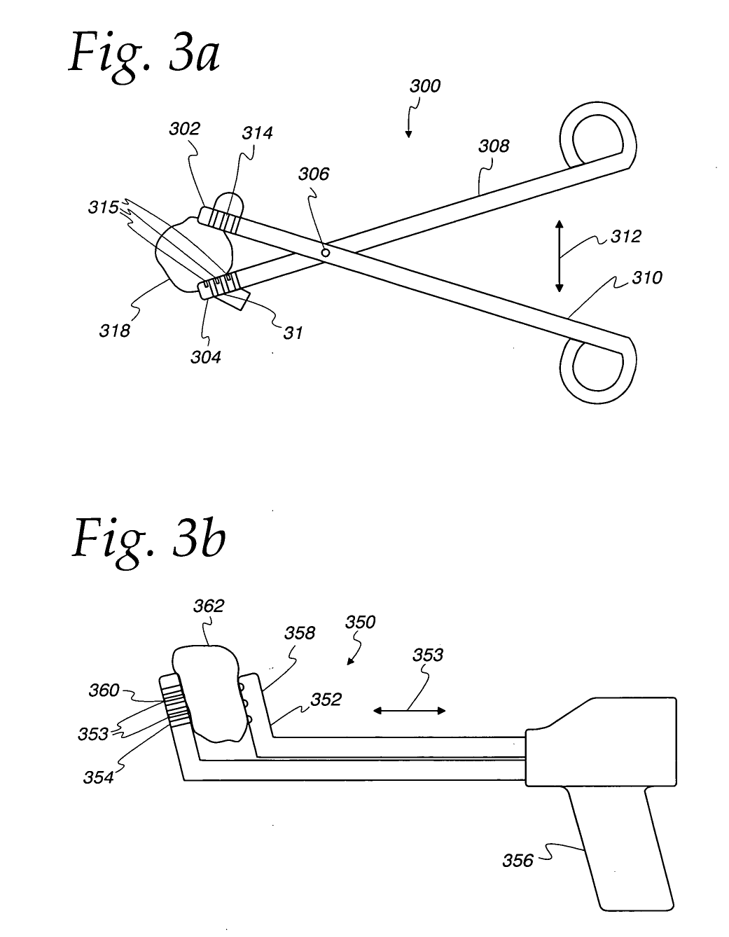

Referring now to FIG. 3a, one example of a clamping arrangement 300 that uses a scissors-like configuration is described. An upper member 302 is connected to a lower member 304 at a pivot point 306 that allows the members 302 and 304 to operate as a scissors. That is, a user at handles 308 and 310 can open and close the scissors-like arrangement by moving the handles 308 and 310 in the direction of an arrow 312.

A plurality of electrodes 314 are positioned on the upper member 302. A plurality of thermocouples 316 and return electrodes 315 are placed on the lower member. The return electrodes 315 are provided with a return electrical path to an ablation system. The return path is electrically isolated from the electrical path that is used to provide RF energy to the electrodes 314. The two members are positioned about a portion of tissue 318. RF energy is applied to the electrodes 314 to perform ablation on the tissue 318.

As mentioned, the lower member 304 has a plurality of thermocouples 316 that may be aligned substantially directly across from one of the electrodes 304. However, it will be understood that two or more electrodes may be aligned to one thermocouple and that two or more thermocouples may be aligned to a single electrode. The return electrodes 315 are provided with a return electrical path. The return path is electrically isolated from the electrical path that is used to provide RF energy to the electrodes, which uses a scissors-configuration clamping device.

In one example of the operation of the system of FIG. 3a, the operator positions the device 300 with the two members 302 and 304 between the tissue 318 to be ablated. The operator maneuvers the pair of scissors to be positioned around the tissue 318. Once in place, RF energy can be applied to the tissue 318.

Referring now to FIG. 3b, an example of a clamping arrangement 350 that is in the form of a C-clamp is described. Two members 352 and 354 are positioned and fixed parallel to each other. A handle 356 is used to move one or both of the members 352 or 354 back and forth in the direction of arrow 353. In one example, the member 352 may be fixed and the member 354 may be moved by the handle 356. In another example, both of the members 352 and 354 are moveable by the handle 356.

A plurality of electrodes 358 are positioned on the member 352. A plurality of thermocouples 360 and return electrodes 353 are placed on the member 354. The return electrodes 353 are provided with a return electrical path. The return path is electrically isolated from the electrical path that is used to provide RF energy to the electrodes 358. The two members 352 and 354 are positioned about a portion of tissue 362. RF energy is applied to the electrodes 358 to perform ablation on the tissue 362.

As mentioned, the second member 354 has a plurality of thermocouples 360 that may be aligned substantially directly across from one of the electrodes 358. However, it will be understood that two or more electrodes may be aligned to one thermocouple and that two or more thermocouples may be aligned to a single electrode. The thermocouples are provided with a return electrical path. The return path is electrically isolated from the electrical path that is used to provide RF energy to the electrodes.

In one example of the operation of the system of FIG. 3b, the operator places the device 350 around the tissue 362 to be ablated. The operator can use the handle 356 to adjust one of the members 352 or 354 so that the electrodes 358 are in close proximity to the tissue 362. Once the electrodes 358 and the thermocouples 360 are aligned, the operator can apply RF energy to the electrodes 358 and the tissue 362 can be ablated.

It will be understood that the flex circuit techniques described herein can be used for both the lower and upper members of the clamping device. Thus, thermocouples and return electrodes may be added to the embodiments described above.

Those skilled in the art will recognize that a wide variety of modifications, alterations, and combinations can be made with respect to the above described embodiments without departing from the spirit and scope of the invention, and that such modifications, alterations, and combinations are to be viewed as being within the scope of the invention.

What is claimed is:

1. A device for ablating tissue in the human body comprising:
a first member having a plurality of electrodes positioned thereupon, the electrodes being adapted to provide energy to ablate human tissue; and
a second member in communication with the first member, the second member having a plurality of temperature sensing elements positioned thereupon, each of the temperature sensing elements being positioned so as to be substantially directly opposite from at least one of the plurality of electrodes on the first member.

2. The device of claim 1 wherein the first and second members comprise a mechanical configuration that is substantially parallel and opposite from each other.

3. The device of claim 1 wherein each of the plurality of electrodes comprises an electrode selected from a group comprising: an etched electrode and a non-etched electrode.

4. The device of claim 1 wherein temperature information is obtained by the temperature sensing elements and is provided to an evaluation system.

5. The device of claim 1 wherein impedance information is obtained by electrodes and provided to the evaluation system.

6. The device of claim 1 wherein the first and second members comprise a configuration selected from a group comprising: a C-clamp configuration and a scissors configuration.

7. The device of claim 1 wherein the temperature sensing elements are chosen from a group comprising thermocouples and thermistors.

8. A method for ablating tissue in the human body comprising:
positioning a first member at a first side of a portion of human tissue, the first member having a plurality of electrodes disposed thereupon;
positioning a second member at a second and opposite side of the human tissue, the second member having a plurality of thermocouples disposed thereupon;
aligning the first member with the second member such that each of the thermocouples on the second member is positioned across the tissue from and in the general vicinity of at least one electrode for recording the temperature of the tissue adjacent to the at least one electrode;
supplying RF energy to the plurality of electrodes; and
providing a return electrical path for the plurality of electrodes, the path being electrically isolated from the RF energy supplied to the plurality of electrodes and terminating at an ablation monitoring and RF energy delivery system that responsively adjusts an amount of RF energy delivered to the plurality of electrodes based upon the temperature.

9. The method of claim 8 wherein said first and second members comprises are aligned in a mechanical configuration substantially parallel and opposite from each other.

10. The method of claim 8 further comprising obtaining temperature information from the plurality of thermocouples and providing the temperature information to an evaluation system.

11. The method of claim 8 further comprising positioning the thermocouples on the exterior of the second member so as to come in direct contact with the portion of the human tissue.

12. The method of claim 8 wherein the aligning of the first and second members comprises aligning the first and second members in a configuration selected from a group comprising: a C-clamp configuration and a scissors configuration.

13. A device for ablating tissue in the human body comprising:
a first member having a plurality of electrodes positioned thereupon, the electrodes provided with radio frequency (RF) energy from an ablation monitoring and RF energy delivery system and the first member being positioned at a first side of a portion of human tissue; and
a second member having a plurality of thermocouples, the second member being positioned at a second and opposite side of the portion of the human tissue from the first member such that each of the plurality of electrodes of the first member are positioned across the tissue from and in the general vicinity of at least one of the plurality of thermocouples for recording the temperature of the tissue adjacent to the at least one electrode, wherein the thermocouples are positioned on an exterior surface of the second member so as to be in direct contact with a facing portion of the human tissue, and wherein the second member is coupled to a return path to the ablation monitoring and RF energy delivery system, the path being electrically isolated from the RF energy provided to the plurality of electrodes and wherein the ablation monitoring and RF energy delivery system responsively adjusts an amount of RF energy supplied to the plurality of electrodes based upon the temperature.

14. The device of claim 13 wherein the first and second members comprise a mechanical configuration that is substantially parallel and opposite from each other.

15. The device of claim 13 wherein each of the plurality of electrodes comprises an electrode selected from a group comprising: an etched electrode and a non-etched electrode.

16. The device of claim 13 wherein the temperature information obtained by the thermocouples is provided to an evaluation system.

17. The device of claim 13 wherein the impedance information obtained by the electrodes is provided to an evaluation system.

18. The device of claim 13 wherein the first and second members comprise a configuration selected from a group comprising: a C-clamp configuration and a scissors configuration.

19. A system for ablating tissue in the human body comprising:
an ablation monitoring and RF energy delivery system;
a first member coupled to the ablation monitoring and RF energy delivery system, the first member having a plurality of electrodes positioned so as to be in direct contact with a first side of a portion of human tissue, the plurality of electrodes receiving radio frequency (RF) energy from the ablation monitoring and RF energy delivery system; and
a second member coupled to the ablation monitoring and RF energy delivery system and having a plurality of thermocouples positioned thereupon, the second member being positioned across the tissue from and in the general vicinity of at least one of the plurality of electrodes for recording the temperature of the tissue adjacent to the at least one electrode, wherein the plurality of thermocouples are positioned on an exterior surface of the second member so as to be in direct contact with a facing portion of the human tissue, the second member being connected via a return path to the ablation monitoring and RF energy delivery system, the return path being electrically isolated from the RF energy provided to the plurality of electrodes on the first member, the temperature being used by the ablation monitoring and RF energy delivery system to potentially adjust an amount of the RF energy delivered to the plurality of electrodes.

20. The system of claim 19 wherein the first and second member comprise a mechanical configuration substantially parallel and opposite from each other.

21. The system of claim 19 wherein each of the plurality of thermocouples are comprised of gold.

22. The system of claim 19 wherein each of the plurality of electrodes comprises an electrode selected from a group comprising an etched electrode and a non-etched electrode.

23. The system of claim 19 wherein the first member is formed from a flex circuit.

24. The system of claim 19 wherein the first and second members comprise a configuration selected from a group comprising a C-clamp configuration and a scissors configuration.
